(19) United States
(12) Patent Application Publication
(10) Pub. No.: US 2007/0031343 A1
(43) Pub. Date: Feb. 8, 2007

(54) FLAVORED MEDICINAL INHALANT

(76) Inventors: Charles A. Bonfour III, St. Augustine, FL (US); Patricia A. Bonfour, St. Augustine, FL (US)

Correspondence Address: MARK YOUNG, P.A., 12086 FORT CAROLINE ROAD, UNIT 202, JACKSONVILLE, FL, 32225, US

(21) Appl. No.: 11/460,990

(22) Filed: Jul. 30, 2006

Related U.S. Application Data
(60) Provisional application No. 60/705,165, filed on Aug. 4, 2005.

(51) Int. Cl. A61K 36/534; A61K 36/53; A61L 9/04

(52) U.S. Cl. 424/45; 424/745; 424/747

(57) ABSTRACT

A flavored medicinal inhalant includes a medicant, a carrier and an effective amount of a flavoring agent. Carriers such as saline or bacteriostatic water may be utilized. Flavoring agents include natural and artificial flavors chosen from synthetic flavor oils and flavoring aromatics, and/or oils, oleo resins and extracts derived from plants, leaves, flowers, fruits and so forth, and combinations thereof, spearmint oil, cinnamon oil, peppermint oil, clove oil, bay oil, thyme oil, cedar leaf oil, oil of nutmeg, oil of sage, and oil of bitter almonds, artificial, natural or synthetic fruit flavors such as vanilla, chocolate, coffee, cocoa and citrus oil, including lemon, orange, grape, lime and grapefruit and fruit essences including apple, pear, peach, strawberry, raspberry, cherry, plum, pineapple, apricot and so forth. The flavoring agent may be provided in a single-use container such as a vile or a bulk multi-use container and introduced into the medicinal inhalant composition before administration.

### RELATED APPLICATION

This application claims the benefit of priority of U.S. Provisional Application 60/705,165, filed Aug. 4, 2005, the entire contents of which are incorporated herein.

### FIELD OF THE INVENTION

This invention generally relates to inhaled medication, and more particularly, to flavored medication administered by inhalation devices and methods.

### BACKGROUND

Certain medications, especially those intended for the treatment of acute and chronic respiratory disorders, are most effective when inhaled directly into the lungs. Numerous pharmaceutical products are available for use as aerosols administered from metered dose inhalers. For example, bronchodilators are used in the treatment of bronchospasm and mucosal edema. Antibiotic aerosols are used to treat bronchial infections, anti-inflammatory steroids are used in the treatment of asthma, antifoaming agents are used in the treatment of fulminant pulmonary edema, and cromolyn sodium is used to control allergic asthma.

Illustratively, a commonly used medicinal inhalant is a bronchodilator, i.e., a medication intended to improve bronchial airflow. Treatment of bronchial asthma is the most common application of these drugs. They are also intended to help expand the airways and improve the breathing capacity of patients with emphysema, pneumonia and bronchitis.

Another example of an inhaled medication is a mucolytic agent. Illustratively, acetylcysteine-based mucolytic agents available in aqueous solutions, such as Mead Johnson & Company's Mucomyst®, are antioxidant drugs used to reduce the thickness of mucus and ease its removal.

While medicinal inhalants have proven to be effective for their respective therapies, such as treatment of acute and chronic respiratory disorders, they suffer unpleasant side effects. One such side effect is an unusual or disagreeable taste. Patients frequently describe the taste of bronchodilators as metallic, bitter or acerbic. Related side effects include dry mouth, irritated throat, oropharyngeal numbness and altered taste sensation. Acetylcysteine has a very foul sulfur odor which leaves a bad taste, and may even cause nausea and vomiting.

Accordingly, a need exists for a flavorant that is compatible with medicinal inhalants and substantially improves the palatability of the inhalant without adversely impacting efficacy. The invention is directed to overcoming one or more of the problems and solving one or more of the needs as set forth above.

While taste is limited to sweet, sour, bitter, salty, umami, and other basic tastes, the odors of substances are potentially limitless. A flavor, therefore, can be easily altered by changing its smell while keeping its taste similar. For this reason, although the terms “flavor,” “flavoring” or “flavorant” are employed herein to denote additives that alter the basic taste of medicinal inhalants, as used herein such terms are intended to further encompass compounds that alter flavor through the sense of smell.

### SUMMARY OF THE INVENTION

In one aspect of the invention, a flavored medicinal inhalant is provided. The flavored medicinal inhalant is comprised of a medicant, a carrier and an effective amount of a flavoring agent. Liquid and aerosol carriers suitable for medicinal inhalant compositions, such as saline or bacteriostatic water, may be utilized. Aerosol formulations may include fluorinated alkane propellants, surfactants and co-solvents, and may be filled into an aerosol container which is then closed by a suitable metering valve and pressurized with aerosol propellant. Flavoring agents that can be used, include natural and artificial flavors chosen from synthetic flavor oils and flavoring aromatics, and/or oils, oleo resins and extracts derived from plants, leaves, flowers, fruits and so forth, and combinations thereof, spearmint oil, cinnamon oil, peppermint oil, clove oil, bay oil, thyme oil, cedar leaf oil, oil of nutmeg, oil of sage, and oil of bitter almonds, artificial, natural or synthetic fruit flavors such as vanilla, chocolate, coffee, cocoa and citrus oil, including lemon, orange, grape, lime and grapefruit and fruit essences including apple, pear, peach, strawberry, raspberry, cherry, plum, pineapple, apricot and so forth. The amount may be varied in order to obtain the result desired in the final composition. Amounts of about 0.1 to about 30 percent by weight are useable, with amounts of about 1 to about 10 percent by weight being preferred, and amounts from about 1 to about 5 percent by weight being particularly preferred.

In another aspect of the invention, various methods and means for introducing the flavoring agent into the medicinal inhalant may be employed. An effective amount of flavoring agent may be provided in a single-use container such as a vile and introduced into the medicinal inhalant composition before administration. Alternatively, the flavoring agent may be stored in a bulk multi-use container and introduced in determined amounts into the medicinal inhalant composition before administration. As another alternative, the flavoring agent may be added to the medicinal inhalant composition as part of the process of manufacture. As yet another alternative, the flavoring agent may be stored in a separate compartment from the medicinal inhalant and introduced in a metered dose along with the medicinal inhalant during administration.

### DETAILED DESCRIPTION

The invention is not limited to any particular vehicles, solvents, carriers or excipients and is not restricted to any particular methods or apparatus for administration by inhalation. As those skilled in the art will appreciate, many conventional methods and apparatus are available for administering precisely metered doses of medicinal inhalant. Delivery may be achieved by any of various inhalant approaches, including liquid nebulizers, pump or squeeze-actuated nebulized or atomized spray dispensers, aerosol-based metered dose inhalers, and any other systems suitable for delivery of medicinal inhalants.

Additionally, there are many liquid and aerosol carriers suitable for medicinal inhalant compositions according to principles of the invention. Specific carriers employed may be varied depending upon factors such as size of the subject being treated, treatment dose, and the like. Non-limiting examples of suitable carrier compositions comprise a pharmaceutically acceptable inhalant solvent or carrier, e.g., saline or bacteriostatic water. Aerosol formulations may include fluorinated alkane propellants, surfactants and co-solvents, and may be filled into an aerosol container which is then closed by a suitable metering valve and pressurized with aerosol propellant.

Flavoring agents are added to enhance the acceptance of the medicine. An effective amount of a flavoring agent is added to the medicinal inhalant composition. In a particular preferred implementation, the flavoring agent is added to the carrier (e.g., saline), which may then be mixed with the medication to be inhaled. When combined with the medication to be inhaled, the flavored saline improves palatability of the medicinal inhalant.

A wide array of pharmaceutically compatible flavoring agents may be utilized. Such flavoring agents include natural and artificial flavors chosen from synthetic flavor oils and flavoring aromatics, and/or oils, oleo resins and extracts derived from plants, leaves, flowers, fruits and so forth, and combinations thereof. Non-limiting representative flavoring agents include: almond oil, amaretto flavor, anise oil, natural apple flavor, apricot flavor, banana creme flavor, bavarian creme flavor, vanilla extract, black walnut flavor, blackberry flavor, blueberry flavor, brandy flavor, bubble gum flavor, butter flavor, butter rum flavor, butterscotch flavor, caramel flavor, champagne flavor, cheesecake flavor, cherry flavor, chocolate flavor, chocolate hazelnut flavor, cinnamon oil clove oil, natural coconut flavor, coffee flavor, cotton candy flavor, cran-raspberry flavor, cranberry flavor, creme dementhe flavor, eggnog flavor, English toffe flavor, chili flavor, tart & sour flavor, ginger oil, natural grape flavor, grapefruit oil, pink, natural guava flavor, honey flavor, horehound flavor, coffee flavor, lemon oil, natural lemonade flavor, licorice flavor, lime oil, natural mango flavor, maple flavor, marshmallow flavor, menthol, eucalyptus flavor, mint chocolate chip flavor, nutmeg oil, natural orange cream flavor, orange oil, natural peach flavor, pecan flavor, peppermint oil, pina colada flavor, pineapple flavor, pistachio flavor, plum flavor, praline and cream flavor, praline flavor, pumpkin flavor, raspberry flavor, red licorice flavor, root beer flavor, salt water taffy flavor, sassafras flavor, spearmint oil, natural strawberry flavor, tangerine oil, natural teaberry flavor, tropical punch flavor, tutti-frutti (passion fruit) flavor, vanilla butternut flavor, watermelon flavor, and wintergreen oil, all of which are available from food industry suppliers, such as LorAnn Oils, Inc., 4518 Aurelius Road Lansing, Mich. 48909. These flavorings can be used individually or in admixture.

The amount of flavoring agent employed is a matter of preference subject to such factors as flavoring agent, medicinal inhalant composition, and intensity desired. Thus, the amount may be varied in order to obtain the result desired in the final composition. Such variations are within the capabilities of those skilled in the art without the need for undue experimentation. In general, amounts of about 0.0001% to about 30% percent by weight are useable, with amounts of about 0.0001% to about 10% percent by weight being preferred, and amounts from about 0.0001% to about 5% percent by weight being particularly preferred. Also effective are amounts of about 0.0001% to about 10% percent by volume, with amounts of about 0.0001% to about 5% percent by volume being preferred. Those skilled in the art will appreciate that a palatable concentration of flavoring agent may be readily determined without undue experimentation for a determined medicinal inhalant, and that the concentration may be varied according to the taste of the medication component, concentration (i.e., intensity) of the flavoring agent, and preferences of the user.

Various methods and means for introducing the flavoring agent into the medicinal inhalant may be employed. An effective amount of flavoring agent may be provided in a single-use container and introduced into the medicinal inhalant composition before administration. Alternatively, the flavoring agent may be stored in a bulk multi-use container and introduced in determined amounts into the medicinal inhalant composition before administration. As another alternative, the flavoring agent may be added to the medicinal inhalant composition as part of the process of manufacture. As yet another alternative, the flavoring agent may be stored in a separate compartment from the medicinal inhalant and introduced in a metered dose along with the medicinal inhalant during administration.

While an exemplary embodiment of the invention has been described, it should be apparent that modifications and variations thereto are possible, all of which fall within the true spirit and scope of the invention. With respect to the above description then, it is to be realized that the optimum relationships for the components of the invention and steps of the process, including variations in form, function and manner of operation, are deemed readily apparent and obvious to one skilled in the art, and all equivalent relationships to those described in the specification are intended to be encompassed by the present invention. The above description is illustrative of modifications that can be made without departing from the present invention, the scope of which is to be limited only by the following claims. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents are intended to fall within the scope of the invention as claimed.

1. A flavored medicinal inhalant comprising a medicant, a carrier and an effective amount of a flavoring agent.

2. A flavored medicinal inhalant according to claim 1, wherein the carrier comprises saline.

3. A flavored medicinal inhalant according to claim 1, wherein the carrier comprises bacteriostatic water.

4. A flavored medicinal inhalant according to claim 1, further comprising a fluorinated alkane propellant.

5. A flavored medicinal inhalant according to claim 1, said flavored medicinal inhalant being contained in an aerosol container closed by a metering valve and pressurized with aerosol propellant.

6. A flavored medicinal inhalant according to claim 1, wherein the flavoring agent comprises a flavorant from the group consisting of spearmint oil, cinnamon oil, peppermint oil, menthol, clove oil, bay oil, thyme oil, cedar leaf oil, oil of nutmeg, oil of sage, and oil of almonds.

7. A flavored medicinal inhalant according to claim 1, wherein the flavoring agent comprises a flavorant from the group of natural fruit extracts consisting of vanilla, chocolate, coffee, cocoa, lemon, orange, grape, lime, grapefruit, apple, pear, peach, strawberry, raspberry, cherry, plum, pineapple and apricot.

8. A flavored medicinal inhalant according to claim 1, wherein the flavoring agent comprises a flavorant from the group of synthetic fruit flavored additives consisting of vanilla, chocolate, coffee, cocoa, lemon, orange, grape, lime, grapefruit, apple, pear, peach, strawberry, raspberry, cherry, plum, pineapple and apricot.

9. A flavored medicinal inhalant according to claim 1, wherein the flavoring agent comprises 0.1 to 30 percent by weight of the flavored medicinal inhalant.

10. A flavored medicinal inhalant according to claim 1, wherein the flavoring agent comprises 0.1 to 10 percent by weight of the flavored medicinal inhalant.

11. A flavored medicinal inhalant according to claim 1, wherein the flavoring agent comprises 0.1 to 5 percent by weight of the flavored medicinal inhalant.

12. A flavoring agent for flavoring a medicinal inhalant, said flavoring agent comprising an effective amount of flavoring agent for a single dose of medicinal inhalant, said effective amount of flavoring agent being contained in a disposable container.

13. A flavored medicinal inhalant comprising a medicant, a carrier and an effective amount of a flavoring agent, wherein the carrier comprises saline, and the flavoring agent comprises a flavorant from the group consisting of spearmint oil, cinnamon oil, peppermint oil, menthol, clove oil, bay oil, thyme oil, cedar leaf oil, oil of nutmeg, oil of sage, oil of almonds, vanilla, chocolate coffee cocoa, lemon, orange grape, lime, grapefruit, apple, pear, peach, strawberry, raspberry, cherry, plum, pineapple, apricot, almond oil, amaretto flavor, anise oil, natural apple flavor, apricot flavor, banana creme flavor, bavarian creme flavor, vanilla extract, black walnut flavor, blackberry flavor, blueberry flavor, brandy flavor, bubble gum flavor, butter flavor, butter rum flavor, butterscotch flavor, caramel flavor, champagne flavor, cheesecake flavor, cherry flavor, chocolate flavor, chocolate hazelnut flavor, cinnamon oil clove oil, natural coconut flavor, coffee flavor, cotton candy flavor, cran-raspberry flavor, cranberry flavor, creme dementhe flavor, eggnog flavor, English toffe flavor, chili flavor, tart & sour flavor, ginger oil, natural grape flavor, grapefruit oil, pink, natural guava flavor, honey flavor, horehound flavor, coffee flavor, lemon oil, natural lemonade flavor, licorice flavor, lime oil, natural mango flavor, maple flavor, marshmallow flavor, menthol, eucalyptus flavor, mint chocolate chip flavor, nutmeg oil, natural orange cream flavor, orange oil, natural peach flavor, pecan flavor, peppermint oil, pina colada flavor, pineapple flavor, pistachio flavor, plum flavor, praline and cream flavor, praline flavor, pumpkin flavor, raspberry flavor, red licorice flavor, root beer flavor, salt water taffy flavor, sassafras flavor, spearmint oil, natural strawberry flavor, tangerine oil, natural teaberry flavor, tropical punch flavor, tutti-frutti flavor, vanilla butternut flavor, watermelon flavor, and wintergreen oil.

14. (canceled)

15. (canceled)

16. (canceled)

17. A flavored medicinal inhalant according to claim 12, wherein the flavoring agent comprises 0.1 to 30 percent by weight of the flavored medicinal inhalant.

18. A flavored medicinal inhalant according to claim 12, wherein the flavoring agent comprises 0.1 to 10 percent by weight of the flavored medicinal inhalant.

19. A flavored medicinal inhalant according to claim 12, wherein the flavoring agent comprises 0.1 to 5 percent by weight of the flavored medicinal inhalant.
